(19) United States
(12) Patent Application Publication
(10) Pub. No.: US 2026/0061391 A1
(43) Pub. Date: Mar. 5, 2026

(54) COPPER EMBEDDED POLYURETHANE FOAM FOR MERCURY REMOVAL

(71) Applicant: UOP LLC, Des Plaines, IL (US)

(72) Inventors: Nilesh Tekawade, Haryana (IN); Gautam Pandey, Harayana (IN); Shweta Sharma, Haryana (IN); Manasjyoti Nath, Haryana (IN)

(21) Appl. No.: 19/282,039

(22) Filed: Jul. 28, 2025

Related U.S. Application Data
(60) Provisional application No. 63/687,811, filed on Aug. 28, 2024.

(51) Int. Cl. B01J 20/02; B01D 53/02; B01J 20/28; B01J 20/30; C07C 7/12

(52) U.S. Cl. CPC B01J 20/0237; B01D 53/02; B01J 20/0285; B01J 20/28011; B01J 20/28045; B01J 20/28085; B01J 20/28095; B01J 20/3007; B01J 20/3064; B01J 20/3085; C07C 7/12; B01D 2253/1128; B01D 2253/202; B01D 2253/25; B01D 2256/24; B01D 2257/602

(57) ABSTRACT

Adsorbents for removing contaminants from hydrocarbon streams are described. The adsorbents comprise polyurethane foam comprising a metal compound. The polyurethane foam can be rigid or flexible and has a structure comprising a combination of open cells and closed cells. Methods of removing contaminants from hydrocarbon streams using the adsorbent are also described.

### RELATED APPLICATIONS

This application claims priority to U.S. Provisional Patent Application Ser. No. 63/687,811, filed on Aug. 28, 2024, the entirety of which is incorporated herein by reference.

### BACKGROUND

Copper-based guard bed adsorbents are versatile adsorbents for removal of elemental mercury in natural gas purification. Basic copper carbonate (BCC) is the active ingredient which facilities the removal of mercury. In this process, BCC is sulfided to form the active state, copper sulfide, which acts as a scavenger for mercury. Typical current commercially adsorbents for mercury have alumina as the base material. The porosity of these materials is in the micro to nano region because of poring limitation and strength contraction. Reduced porosity often leads to the formation of an “egg-shell” due to diffusion limitations. Experimental data have shown that mercury is adsorbed only on the outer surface of the adsorbents, up to 80 microns, resulting in the formation of “egg-shell. The active copper sites at the inner core remain unused, which limits the mercury removal capacity. Additionally, loss of the structural integrity of adsorbent after a liquid carryover has been reported. Current adsorbents are not capable of handling periodic liquid carryover, causing an increase in high pressure drop along the bed which leads to mechanical failure.

Therefore, there is a need for an adsorbent for the removal of mercury and other contaminants that maintains the porosity and structural integrity of the adsorbent.

### DESCRIPTION

The present invention meets this need by providing an adsorbent for removing a variety of contaminants from hydrocarbon streams. The adsorbent comprises a metal compound dispersed on polyurethane foam to overcome the limitations of the current guard bed products. The polyurethane foam has a structure comprising a combination of open cells and closed cells. The presence of macro pores in the foam sample would mitigate the resistance issue of mercury and other contaminants and thus, the formation of the “egg-shell.” Therefore, all the active metal sites would easily be accessible, facilitating better contaminant removal. Improving the mass transfer zone will enhance metal utilization and increase contaminant uptake capacity. Another advantage of metal loaded foam samples is that, unlike GB products, they can tolerate a much higher level of moisture present in the feed. In addition, the weight of the adsorbent required for getting the same performance can be reduced because the density of the foam is significantly less than traditional adsorbents. This approach is a new and unique way to immobilize the active metal component within polyurethane back bone and to use it for contaminant removal from any fluid stream.

One aspect of the invention is an adsorbent for removing a contaminant from a hydrocarbon stream. In one embodiment, the adsorbent comprises polyurethane foam comprising a metal compound, wherein the polyurethane foam comprises 5% to 95% closed cells, or 5% to 90%, or 5% to 85%, or 5% to 80%, or 5% to 75%, or 5% to 70%, or 5% to 65%, or 5% to 60%, or 10% to 95%, or 10% to 90%, or 10% to 85%, or 10% to 80%, or 10% to 75%, or 10% to 70%, or 10% to 65%, or 10% to 60%, or 20% to 95%, or 20% to 90%, or 20% to 85%, or 20% to 80%, or 20% to 75%, or 20% to 70%, or 20% to 65%, or 20% to 60%, or 30% to 95%, or 30% to 90%, or 30% to 85%, or 30% to 80%, or 30% to 75%, or 30% to 70%, or 30% to 65%, or 30% to 60%, or 40% to 95%, or 40% to 90%, or 40% to 85%, or 40% to 80%, or 40% to 75%, or 40% to 70%, or 40% to 65%, or 40% to 60%, or 50% to 95%, or 50% to 90%, or 50% to 85%, or 50% to 80%, or 50% to 75%, or 50% to 70%, or 50% to 65%, or 50% to 60%.

In some embodiments, the polyurethane foam can be a flexible foam. In some embodiments, the polyurethane foam can be a rigid foam.

In some embodiments, the polyurethane foam has a density in a range of 10 to 400 kg/m³, or 10 to 350 kg/m³, or 10 to 300 kg/m³, or 10 to 250 kg/m³, or 10 to 200 kg/m³, or 10 to 150 kg/m³, or 10 to 120 kg/m³.

In some embodiments, the polyurethane foam has an average pore size of 1 μm to 900 μm (measured using SEM), or 1 μm to 800 μm, or 1 μm to 700 μm, or 1 μm to 600 μm, or 1 μm to 500 μm, or 1 μm to 400 μm, or 1 μm to 300 μm, or 1 μm to 200 μm, or 1 μm to 100 μm, or 10 μm to 900 μm, or 10 μm to 800 μm, or 10 μm to 700 μm, or 10 μm to 600 μm, or 10 μm to 500 μm, or 10 μm to 400 μm, or 10 μm to 300 μm, or 10 μm to 200 μm, or 10 μm to 100 μm, or 20 μm to 900 μm, or 20 μm to 800 μm, or 20 μm to 700 μm, or 20 μm to 600 μm, or 20 μm to 500 μm, or 20 μm to 400 μm, or 20 μm to 300 μm, or 20 μm to 200 μm, or 20 μm to 100 μm, or 30 μm to 900 μm, or 30 μm to 800 μm, or 30 μm to 700 μm, or 30 μm to 600 μm, or 30 μm to 500 μm, or 30 μm to 400 μm, or 30 μm to 300 μm, or 30 μm to 200 μm, or 30 μm to 100 μm, or 40 μm to 900 μm, or 40 μm to 800 μm, or 40 μm to 700 μm, or 40 μm to 600 μm, or 40 μm to 500 μm, or 40 μm to 400 μm, or 40 μm to 300 μm, or 40 μm to 200 μm, or 40 μm to 100 μm, or 45 μm to 900 μm, or 45 μm to 800 μm, or 45 μm to 700 μm, or 45 μm to 600 μm, or 45 μm to 500 μm, or 45 μm to 400 μm, or 45 μm to 300 μm, or 45 μm to 200 μm, or 45 μm to 100 μm, or 50 μm to 900 μm, or 50 μm to 800 μm, or 50 μm to 700 μm, or 50 μm to 600 μm, or 50 μm to 500 μm, or 50 μm to 400 μm, or 50 μm to 300 μm, or 50 μm to 200 μm, or 50 μm to 100 μm, or 75 μm to 900 μm, or 75 μm to 800 μm, or 75 μm to 700 μm, or 75 μm to 600 μm, or 75 μm to 500 μm, or 75 μm to 400 μm, or 75 μm to 300 μm, or 75 μm to 200 μm, or 75 μm to 100 μm, or 100 μm to 900 μm, or 100 μm to 800 μm, or 100 μm to 700 μm, or 100 μm to 600 μm, or 100 μm to 500 μm, or 100 μm to 400 μm, or 100 μm to 300 μm, or 100 μm to 200 μm.

In some embodiments, the metal compound comprises a metal of Groups 2 and 7-12 of the Periodic Table, or combinations thereof. In some embodiments, the metal compound comprises a compound of Cu, Mn, Zn, Fe, Ca, Co, Ni, or combinations thereof. One or more metal compounds can be used. For example, a bimetallic foam can be made using two different metals, and a trimetallic foam can be made using three different metals.

In some embodiments, the metal compound comprises a carbonate, an oxide, a sulfide, or combinations thereof of the metal.

In some embodiments, the metal compound is present in an amount of 1% to 65%, or 1% to 60%, or 1% to 55%, or 1% to 50%, or 1% to 45%, or 1% to 40%, or 1% to 35%, or 1% to 30%, or 1% to 25%, or 1% to 20%, or 1% to 16%, or 1% to 15%, or 5% to 65%, or 5% to 60%, or 5% to 55%, or 5% to 50%, or 5% to 45%, or 5% to 40%, or 5% to 35%, or 5% to 30%, or 5% to 25%, or 5% to 20%, or 5% to 16%, or 5% to 15%, or 10% to 65%, or 10% to 60%, or 10% to 55%, or 10% to 50%, or 10% to 45%, or 10% to 40%, or 10% to 35%, or 10% to 30%, or 10% to 25%, or 10% to 20%, or 10% to 16%, or 10% to 15%, or 12% to 65%, or 12% to 60%, or 12% to 55%, or 12% to 50%, or 12% to 45%, or 12% to 40%, or 12% to 35%, or 12% to 30%, or 12% to 25%, or 12% to 20%, or 12% to 16%, or 12% to 15%, or 20% to 65%, or 20% to 60%, or 20% to 55%, or 20% to 50%, or 20% to 45%, or 10% to 40%, or 20% to 35%, or 20% to 30%, or 20% to 25%.

In some embodiments, the metal compound comprises copper carbonate, sulfided copper (II) oxide, copper (I) oxide, copper (I) sulfide, copper (II) sulfide, CuS (Covellite), CuS (Tenorite), or combinations thereof, and the metal compound is present in an amount of 1% to 65%.

A variety of contaminants can be removed using the adsorbent of the present invention. In some embodiments, the contaminant may comprise mercury, sulfur, arsenic, carbon monoxide, carbon dioxide, or combinations thereof.

The adsorbent can be used to remove contaminants from a variety of hydrocarbon streams. In some embodiments, the hydrocarbon stream comprises a gas stream, a liquid stream, or a stream comprising a mixture of gas and liquid.

In some embodiments, the hydrocarbon stream comprises natural gas, or combinations thereof.

Metal loaded polyurethane foam can easily be prepared with a minor modification to the conventional foam making procedure. The metal compounds are included in the polyol premix which comprises the polyol, the metal compounds (one or more, e.g., two or three), the catalyst, and the blowing agent. The polyol premix is mixed together, combined with isocyanate, and further mixed. The mixture is poured into a mold and allowed to cure.

Any suitable blowing agent can be used. Suitable blowing agents include, but are not limited to, 1-HCFO (chloro-3,3,3-trifluorpropene (1233ZD)), HFC (1,1,1,3,3-pentafluoropropane), pentane, or combinations thereof. Suitable pentanes include, but are not limited to, cyclopentane, iso-pentane, n-pentane, or combinations thereof. The blowing agent can be present in an amount of 1 to 25 wt % of the total mixture (polyol premix and isocyanate).

Any suitable polyol can be used. Suitable polyols include, but are not limited to, polyether polyols.

Any suitable isocyanate can be used. Suitable isocyanates include, but are not limited to, methylene diphenyl diisocyanate.

For example, prior to being used for mercury removal applications, BCC loaded foam needs to be sulfided with H₂S to convert BCC to copper sulfide, which is known to be the active phase for mercury removal applications. If the feed contains both sulfur and mercury contaminants, pre-sulfidation may not be required, as copper sulfide can be formed in-situ.

Copper dispersed foam can also be used as a monolith in the mercury removal application, which makes it easy to handle. Due to the higher dispersion of copper dispersed foam, the utilization of active metal can be increased more than 10 times compared to traditional adsorbents. Copper dispersed foam is stable to liquid carry over.

Another aspect of the invention is a method of removing a contaminant from a hydrocarbon stream. In one embodiment, the method comprises contacting the hydrocarbon stream with an adsorbent comprising a polyurethane foam comprising a metal compound, wherein the polyurethane foam comprises 5% to 95% closed cells.

In some embodiments, the method further comprises treating the metal compound with H₂S to convert the metal compound to metal sulfide.

In some embodiments, the polyurethane foam can be a flexible foam. In some embodiments, the polyurethane foam can be a rigid foam.

In some embodiments, the polyurethane foam has a density in a range of 10 to 400 kg/m³, or 10 to 350 kg/m³, or 10 to 300 kg/m³, or 10 to 250 kg/m³, or 10 to 200 kg/m³, or 10 to 150 kg/m³, or 10 to 120 kg/m³.

In some embodiments, the polyurethane foam has a pore size of 1 μm to 900 μm.

In some embodiments, the metal compound comprises a metal of Groups 2 and 7-12 of the Periodic Table, or combinations thereof. In some embodiments, the metal compound comprises a compound of Cu, Mn, Zn, Fe, Ca, Co, Ni, or combinations thereof.

In some embodiments, the metal compound comprises a carbonate, an oxide, a sulfide, or combinations thereof of the metal.

In some embodiments, the metal compound is present in an amount of 1% to 65%.

In some embodiments, the metal compound comprises copper carbonate, sulfided copper (II) oxide, copper (I) oxide, copper (I) sulfide, copper (II) sulfide, CuS (Covellite), CuS (Tenorite), or combinations thereof, and the metal compound is present in an amount of 1% to 65%.

A variety of contaminants can be removed using the adsorbent of the present invention. In some embodiments, the contaminant may comprise mercury, sulfur, arsenic, carbon monoxide, carbon dioxide, or combinations thereof.

The adsorbent can be used to remove contaminants from a variety of hydrocarbon streams. In some embodiments, the hydrocarbon stream comprises a gas stream, a liquid stream, or a stream comprising a mixture of gas and liquid.

In some embodiments, the hydrocarbon stream comprises natural gas, or combinations thereof.

### EXAMPLES

### Example A

The process involves two primary steps. Initially, a polyol premix is prepared by thoroughly combining polyol 10-80%, catalyst 0.1-5%, surfactant 0.2-6%, carbonate and/or oxides of one, two, or three metal salts of Groups 2 or 7-12 of the periodic table (1-65%), and blowing agent 1-25%. This mixture is stirred at 100-500 rpm for 2-15 minutes. The polyol premix is transferred to a beaker, and isocyanate 29-60% is added, and the mixture is stirred for 10-20 seconds at 2000-3000 rpm. The resulting mixture is poured into a mold or box and allowed to cure for 10-15 minutes.

A polyurethane (PU) foam containing 15% copper was produced by reacting 20-30 grams of basic copper carbonate (BCC) with polyurethane precursors. To create a bimetallic PU foam, 20-30 grams of BCC and 5-10 grams of zinc carbonate were reacted with the polyurethane precursors using the same method.

### Example B

Metal-loaded PU foam is sulfided under conditions of 20° C. to 150° C. and 0.2 to 5.0 bar pressure. A gas mixture containing 0.5% to 100% hydrogen sulfide (H₂S) balanced with nitrogen (N₂) or hydrogen (H₂) is flowed through the foam to the completion of H₂S absorption. This process converts metal carbonates or oxides of Groups 2 and 7-12 elements into their corresponding metal sulfides. The sulfur content of the resulting foam is determined using a sulfur analyzer, such as those made by LECO Corp. of St. Joseph, MI.

The copper-loaded and bimetallic copper-zinc-loaded PU foams prepared as described in Example A were sulfided following the outlined procedure. This resulted in total sulfur contents of 4.0-6.5 wt % and 5.0-9.0 wt %, respectively as shown in Table 1.

### Example C

Mercury (Hg) capacity was determined through breakthrough curve analysis in a fixed-bed adsorbent system operating under plug flow conditions. A mercury-containing stream, generated by diluting a saturated Hg stream with nitrogen or sourced from natural gas or liquid, was introduced into the bed containing metal sulfide as the active phase. The Hg concentration ranged from approximately 10 to 8500 μg Hg/m³. Breakthrough curves were obtained by measuring Hg concentrations at the inlet and within the bed segments using atomic fluorescence spectroscopy (Sir Galahad II, PS Analytical of Orpington, Kent, UK). Experiments were conducted at flow rates of approximately 1.0 to 9.0 L/min, and at contact times between 0.1 and 10 seconds.

Finally, the mercury performance of the sulfided foams, as mentioned in example B, were evaluated using the method outlined above and their efficiency is calculated using equation 1 and reported in Table 01.

The Hg removal efficiency level is defined by:

$$\frac{Hg content on inlet - Hg content on outlet}{Hg conteint in inlet} \times 100 \qquad ( 1 )$$

TABLE 1
Results

| Active Metal | | | Closed | Total Sulfur | Hg Removal |
|---|---|---|---|---|---|
| Phase % | Cu % | Zn % | cells, % | wt. % | efficiency % |
| 5 | 5.67 | 0 | 5-50 | 1.0-2.1 | 87.4 |
| 15 | 15.46 | 0 | 17-64 | 4.0-6.5 | 95.3 |
| 45 | 45.01 | 0 | 25-81 | 12.0-19.0 | 99.9 |
| 65 | 65.10 | 0 | 30-90 | 22.0-29.5 | 99.9 |
| 10 | 5.07 | 4.94 | 5-58 | 2.8-5.0 | 94.6 |
| 20 | 15.05 | 5.02 | 15-72 | 5.0-9.0 | 90.8 |
| 35 | 25.12 | 9.86 | 21-84 | 12.0-15.2 | 99.9 |

### SPECIFIC EMBODIMENTS

While the following is described in conjunction with specific embodiments, it will be understood that this description is intended to illustrate and not limit the scope of the preceding description and the appended claims.

A first embodiment of the invention is a composition for removing a contaminant from a hydrocarbon stream comprising polyurethane foam comprising a metal compound, wherein the polyurethane foam comprises 5% to 95% closed cells. An embodiment of the invention is one, any or all of prior embodiments in this paragraph up through the first embodiment in this paragraph wherein the polyurethane foam has a density in a range of 10 to 400 kg/m³. An embodiment of the invention is one, any or all of prior embodiments in this paragraph up through the first embodiment in this paragraph wherein the polyurethane foam has an average pore size of 1 μm to 900 μm. An embodiment of the invention is one, any or all of prior embodiments in this paragraph up through the first embodiment in this paragraph wherein the contaminant comprises mercury, sulfur, arsenic, carbon monoxide, carbon dioxide, or combinations thereof. An embodiment of the invention is one, any or all of prior embodiments in this paragraph up through the first embodiment in this paragraph wherein the metal compound comprises a metal of Groups 2 and 7-12 of the Periodic Table, or combinations thereof. An embodiment of the invention is one, any or all of prior embodiments in this paragraph up through the first embodiment in this paragraph wherein the metal compound comprises a compound of Cu, Mn, Zn, Fe, Ca, Co, Ni, or combinations thereof. An embodiment of the invention is one, any or all of prior embodiments in this paragraph up through the first embodiment in this paragraph wherein the metal compound is present in an amount of 1% to 65 wt % based on total amount of adsorbent. An embodiment of the invention is one, any or all of prior embodiments in this paragraph up through the first embodiment in this paragraph wherein the metal compound comprises a carbonate, an oxide, a sulfide, or combinations thereof of the metal. An embodiment of the invention is one, any or all of prior embodiments in this paragraph up through the first embodiment in this paragraph wherein the hydrocarbon stream comprises a gas stream, a liquid stream, or a stream comprising a mixture of gas and liquid. An embodiment of the invention is one, any or all of prior embodiments in this paragraph up through the first embodiment in this paragraph wherein the hydrocarbon stream comprises natural gas. An embodiment of the invention is one, any or all of prior embodiments in this paragraph up through the first embodiment in this paragraph wherein the polyurethane foam is rigid. An embodiment of the invention is one, any or all of prior embodiments in this paragraph up through the first embodiment in this paragraph wherein the polyurethane foam is flexible. An embodiment of the invention is one, any or all of prior embodiments in this paragraph up through the first embodiment in this paragraph wherein the metal compound comprises copper carbonate, sulfided copper (II) oxide, copper (I) oxide, copper (I) sulfide, copper (II) sulfide, CuS (Covellite), CuS (Tenorite), or combinations thereof, and wherein the metal compound is present in an amount of 1% to 65%.

A second embodiment of the invention is a method of removing a contaminant from a hydrocarbon stream comprising contacting the hydrocarbon stream with an adsorbent comprising a polyurethane foam comprising a metal compound, wherein the polyurethane foam comprises 5% to 95% closed cells An embodiment of the invention is one, any or all of prior embodiments in this paragraph up through the second embodiment in this paragraph wherein the polyurethane foam has a density in a range of 10 to 400 kg/m³. An embodiment of the invention is one, any or all of prior embodiments in this paragraph up through the second embodiment in this paragraph wherein the polyurethane foam has a pore size of 1 μm to 900 μm. An embodiment of the invention is one, any or all of prior embodiments in this paragraph up through the second embodiment in this paragraph wherein the contaminant comprises mercury, sulfur, arsenic, carbon monoxide, carbon dioxide, or combinations thereof. An embodiment of the invention is one, any or all of prior embodiments in this paragraph up through the second embodiment in this paragraph wherein the metal compound comprises a metal of Groups 2 and 7-12 of the Periodic Table. An embodiment of the invention is one, any or all of prior embodiments in this paragraph up through the second embodiment in this paragraph wherein the metal compound comprises a compound of Cu, Mn, Zn, Fe, Ca, Co, Ni, or combinations thereof. An embodiment of the invention is one, any or all of prior embodiments in this paragraph up through the second embodiment in this paragraph wherein the metal compound is present in an amount of 1% to 65%. An embodiment of the invention is one, any or all of prior embodiments in this paragraph up through the second embodiment in this paragraph wherein the metal compound comprises a carbonate, an oxide, a sulfide, or combinations thereof of the metal. An embodiment of the invention is one, any or all of prior embodiments in this paragraph up through the second embodiment in this paragraph wherein the hydrocarbon stream comprises a gas stream, a liquid stream, or a stream comprising a mixture of gas and liquid. An embodiment of the invention is one, any or all of prior embodiments in this paragraph up through the second embodiment in this paragraph wherein the hydrocarbon stream comprises natural gas, or combinations thereof. An embodiment of the invention is one, any or all of prior embodiments in this paragraph up through the second embodiment in this paragraph wherein the polyurethane foam is rigid. An embodiment of the invention is one, any or all of prior embodiments in this paragraph up through the second embodiment in this paragraph wherein the polyurethane foam is flexible. An embodiment of the invention is one, any or all of prior embodiments in this paragraph up through the second embodiment in this paragraph wherein the metal compound comprises copper carbonate, sulfided copper (II) oxide, copper (I) oxide, copper (I) sulfide, copper (II) sulfide, CuS (Covellite), CuS (Tenorite), or combinations thereof, and wherein the metal compound is present in an amount of 1% to 65%.

A third embodiment is method of making an adsorbent for removing a contaminant from a hydrocarbon stream comprising providing a polyol premix comprising a polyol, a metal compound, a catalyst, and a blowing agent; mixing the polyol premix with an isocyanate to form a mixture; pouring the mixture into a mold; and curing the mixture to form a polyurethane foam comprising 5% to 95% closed cells. An embodiment of the invention is one, any or all of prior embodiments in this paragraph up through the third embodiment in this paragraph further comprising treating the metal compound with H₂S to convert the metal compound to metal sulfide. An embodiment of the invention is one, any or all of prior embodiments in this paragraph up through the third embodiment in this paragraph wherein the blowing agents (1-25%) comprises 1-HCFO (chloro-3,3,3-trifluoropropene (1233ZD), HFC (1,1,1,3,3-pentafluoropropane), pentane, or combinations thereof. An embodiment of the invention is one, any or all of prior embodiments in this paragraph up through the third embodiment in this paragraph wherein the pentane comprises cyclopentane, iso-pentane, n-pentane, or combinations thereof.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. An adsorbent for removing a contaminant from a hydrocarbon stream comprising:
polyurethane foam comprising a metal compound, wherein the polyurethane foam comprises 5% to 95% closed cells.

2. The adsorbent of claim 1 wherein the polyurethane foam has a density in a range of 10 to 400 kg/m³, or an average pore size of 1 μm to 900 μm, or both.

3. The adsorbent of claim 1 wherein the contaminant comprises mercury, sulfur, arsenic, carbon monoxide, carbon dioxide, or combinations thereof.

4. The adsorbent of claim 1 wherein the metal in the metal compound comprises a metal of Groups 2 and 7-12 of the Periodic Table, or combinations thereof.

5. The adsorbent of claim 1 wherein the metal in the metal compound comprises Cu, Mn, Zn, Fe, Ca, Co, Ni, or combinations thereof.

6. The adsorbent of claim 1 wherein the metal compound is present in an amount of 1% to 65 wt % based on total weight of the adsorbent.

7. The adsorbent of claim 1 wherein the metal compound comprises a carbonate, an oxide, a sulfide, or combinations thereof of the metal.

8. The adsorbent of claim 1 wherein the polyurethane foam is a rigid foam, or a flexible foam.

9. The adsorbent of claim 1 wherein the metal compound comprises copper carbonate, sulfided copper (II) oxide, copper (I) oxide, copper (I) sulfide, copper (II) sulfide, CuS (Covellite), CuS (Tenorite), or combinations thereof, and wherein the metal compound is present in an amount of 1% to 65%.

10. A method of removing a contaminant from a hydrocarbon stream comprising:
contacting the hydrocarbon stream with an adsorbent comprising a polyurethane foam comprising a metal compound, wherein the polyurethane foam comprises 5% to 95% closed cells.

11. The method of claim 10 wherein the polyurethane foam has a density in a range of 10 to 400 kg/m³, or a pore size of 1 μm to 900 μm, or both.

12. The method of claim 10 wherein the contaminant comprises mercury, sulfur, arsenic, carbon monoxide, carbon dioxide, or combinations thereof.

13. The method of claim 10 wherein the metal in the metal compound comprises a metal of Groups 2 and 7-12 of the Periodic Table, wherein the metal compound comprises a carbonate, an oxide, a sulfide, or combinations thereof of the metal, and wherein the metal compound is present in an amount of 1% to 65%.

14. The method of claim 10 wherein the hydrocarbon stream comprises a gas stream, a liquid stream, or a stream comprising a mixture of gas and liquid.

15. The method of claim 10 wherein the hydrocarbon stream comprises natural gas, or combinations thereof.

16. A method of making an adsorbent for removing a contaminant from a hydrocarbon stream comprising:
providing a polyol premix comprising a polyol, a metal compound, a catalyst, and a blowing agent;
mixing the polyol premix with an isocyanate to form a mixture;
pouring the mixture into a mold; and
curing the mixture to form a polyurethane foam comprising 5% to 95% closed cells.

17. The method of claim 16 further comprising:
treating the metal compound with H₂S to convert the metal compound to metal sulfide.

18. The method of claim 16 wherein the blowing agent comprises 1-HCFO (chloro-3,3,3trifluorpropene), HFC (1,1,1,3,3-pentafluoropropane), pentane, or combinations thereof, and wherein the blowing agent is present in an amount of 1-25%.

19. The method of claim 18 wherein the pentane comprises cyclopentane, iso-pentane, n-pentane, or combinations thereof.

20. The method of claim 16 wherein the polyol comprises a polyether polyol and wherein the isocyanate comprises methylene diphenyl diisocyanate.
